(19) United States
(12) Patent Application Publication
(10) Pub. No.: US 2025/0009833 A1
(43) Pub. Date: Jan. 9, 2025

(54) A COMPOSITION AND ITS USE IN MANUFACTURING A MEDICAMENT FOR TREATMENT OF DIABETES

(71) Applicant: MEI-NAN TUAN, New Taipei City (TW)

(72) Inventor: MEI-NAN TUAN, New Taipei City (TW)

(21) Appl. No.: 18/708,753

(22) PCT Filed: Nov. 11, 2022
(86) PCT No.: PCT/CN2022/131466

§ 371 Date: May 9, 2024

Related U.S. Application Data
(60) Provisional application No. 63/278,397, filed on Nov. 11, 2021.

(51) Int. Cl. A61K 36/74; A61K 36/064; A61K 36/185; A61K 36/23; A61K 36/42; A61K 36/82; A61K 36/87; A61K 38/16; A61P 3/10

(52) U.S. Cl. CPC A61K 36/742; A61K 36/064; A61K 36/185; A61K 36/23; A61K 36/42; A61K 36/82; A61K 36/87; A61K 38/168; A61P 3/10

(57) ABSTRACT

A composition and its use in manufacturing a medicament or supplement for treating or reversing diabetes, particularly type 2 diabetes. The composition comprises Bitter melon fruit extract, Celery seed extract, Bakers yeast cell wall extract, Acerola fruit extract, Grape seed extract, Green tea leaf extract, Hydrolyzed soy protein powder, maybe also comprises Green coffee bean extract.

### FIELD OF THE INVENTION

The present invention relates to a method and composition for treating diabetes.

### BACKGROUND OF THE INVENTION

Diabetes mellitus (DM), also referred to as diabetes, is a metabolic disorder in which there are symptoms of chronic hyperglycemia. Serious long-term complications include cardiovascular disease, stroke, chronic kidney disease, food ulcers and damages to the eyes. There are three types of diabetes mellitus:
(1) Type 1 diabetes, referred to as insulin-dependent diabetes mellitus (IDDM), which results from the pancreas' failure to produce enough insulin due to loss of beta cells;
(2) Type 2 diabetes, referred to as non insulin-dependent diabetes mellitus (NIDDM) or adult-onset diabetes, which begins with insulin resistance, a condition in which cells fail to respond to insulin property;
(3) gestational diabetes, which is the third main form, and occurs when pregnant women without a previous history of diabetes develop high blood sugar levels.

Prevention and treatment strategies include maintaining a healthy diet, regular physical exercise, a normal body weight, and a voiding uses of tobacco. Normally, Type 1 diabetes may be managed with insulin injections, and type 2 diabetes may be treated with medications with or without insulin. However, the treatment for diabetes with insulin would be failed if insulin resistance (IR) occurs in the diabetes patients. Both life style (lack of physical activity) and dietary nutrition (excessive high energy foods) have been implicated in the development of diabetes as a consequence of increased insulin resistance in tissues and reduction in insulin secretion from pancreas beta cells.

It is desirable to develop a new approach for treatment of diabetes.

### BRIEF SUMMARY OF THE INVENTION

It is unexpectedly found in the present invention that a natural supplement provides unexpected better efficacy in treating, even reversing, diabetes, particularly type 2 diabetes, which was confirmed by some in vitro experiments and a clinical trial, illustrating that the supplement could be potentially used to treat or reverse diabetes, particularly type 2 diabetes.

In one aspect, the invention provides a supplement for use in treating or reversing diabetes, particularly type 2 diabetes, which comprises, consists essentially of, or consist of Green coffee (Coffea arabica) bean extract, Bitter melon (Momordica charantia) fruit extract, Celery (Apium graveolens) seed extract, Bakers yeast (Saccharomyces cerevisiae) cell wall extract, Acerola (Malpighia emarginata) fruit extract, Grape (Vitis vinifera) seed extract, Green tea leaf extract, and Hydrolyzed soy protein powder. One example of the supplement is a commercial product, Dibibree® of Geninova Biotech, U.S.A.

In one embodiment of the invention, the supplement comprises of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

In one embodiment of the invention, the supplement consists essentially of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

In one embodiment of the invention, the supplement consists of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

In one example of the invention, the supplement comprises 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

In one example of the invention, the supplement consists essentially of 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

In one example of the invention, the supplement consists of 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

It is confirmed in the invention that the supplement exhibits the efficacy in activating Glucagon-like peptide-1 (GLP-1), inhibiting dipeptidyl peptidase-4 (DPP4), and inhibiting formation of Advanced glycation end products (AGEs)

In another aspect, the invention provides a supplement for manufacturing a medicament for treating or reversing diabetes in a subject, wherein the supplement at a therapeutically effective amount to activate Glucagon-like peptide-1 (GLP-1), inhibit Dipeptidyl peptidase-4 (DPP4) and/or inhibit the formation of Advanced glycation end products (AGEs) in the subject.

In another aspect, the invention provides a supplement for manufacturing a medicament for treating or reversing diabetes.

In one embodiment of the invention, the subject is a type 2 diabetes patient.

In one embodiment of the invention, the subject is an insulin-resistance diabetes patient.

In one yet aspect, the present invention provides a pharmaceutical composition for treating or reversing diabetes in a subject, which comprises Bitter melon (Momordica charantia) fruit extract, Celery (Apium graveolens) seed extract, Bakers yeast (Saccharomyces cerevisiae) cell wall extract, Acerola (Malpighia emarginata) fruit extract, Grape (Vitis vinifera) seed extract, Green tea leaf extract, and Hydrolyzed soy protein powder, based on the total weight of the supplement, respectively, and a pharmaceutically acceptable carrier.

In one embodiment of the invention, the pharmaceutical composition comprises of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

In one embodiment of the invention, the pharmaceutical composition consists essentially of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

In one embodiment of the invention, the pharmaceutical composition consists of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

In one embodiment of the invention, the pharmaceutical composition comprises 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

In one embodiment of the invention, the pharmaceutical composition consists essentially of 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

In one embodiment of the invention, the pharmaceutical composition consists of 12.5 wt % Bitter melon (Momordica charantia) fruit extract, 12.5 wt % Celery (Apium graveolens) seed extract, 12.5 wt % Bakers yeast (Saccharomyces cerevisiae) cell wall extract, 12.5 wt % Acerola (Malpighia emarginata) fruit extract, 12.5 wt % Grape (Vitis vinifera) seed extract, 12.5 wt % Green tea leaf extract, and 12.5 wt % Hydrolyzed soy protein powder, based on the total weight of the supplement, respectively.

In one example of the invention, the supplement or pharmaceutical composition for treating or reversing insulin-independent diabetes, particularly type 2 diabetes, is provided.

In one example of the invention, the supplement or pharmaceutical composition for treating or reversing insulin-resistance diabetes.

In one further yet aspect, the present invention provides a combination or composition for treating or reversing diabetes which comprises a therapeutically effective amount of the supplement set forth in claims 1-4, and an anti-diabetes drug, a substance, a peptide, a protein, or a mixture thereof.

In one example of the invention, the diabetes is type 2 diabetes.

In one example of the invention, the diabetes is insulin-resistance diabetes.

### BRIEF DESCRIPTION OF THE SEVERAL VIEWS OF THE DRAWINGS

FIG. 1 illustrates the overview of the process connecting disease, pathways, and Dibifree signatures through big data analysis.

FIG. 2 provides the Volcano plot demonstrating the gene expression profile of Dibifree, which illustrates significant DEGs of Dibifree treatment in BEAS2B cell. The x axis was log 2 fold change and the y axis was p-value (<0.001). The right side denoted the up-regulation genes of Dibifree while left side was down-regulation. The color scale bar described the p-value.

FIG. 3 shows the results of using the gene expression profile of Dibifree to match the similar perturbagens on CLUE platform.

FIG. 4 shows that Dibifree reverses the diabetic nephropathy signature from GSEA analysis.

FIG. 5 shows that the biological function of Dibifree could be connected to comorbidities and complications of diabetes via (A) DO and (B) DisGeNET databases. The network showed the top 15 associated diseases from Disease Ontology and DisGeNET analysis. The color scale bar indicated the p-value and a dot size was the gene counts.

FIG. 6 shows that the Gene Ontology and KEGG enrichment predict the potential pathways modulated by Dibifree. (A) Pathways were associated to diabetes from GO analysis. The color scale bar described the p-value and x axis was the overlapped gene counts between DEGs of Dibifree and the database. (B) The top 10 pathway from KEGG prediction. The color scale bar indicated the p-value and x axis was the gene counts.

FIG. 7 shows the effects of Dibifree on methylglyoxal induced advanced glycation endproducts (AGEs) formation. (A) AGE formation in the presence of Dibifree, aminoguanadine (AG) and metformin was measured. Data represents as Mean±SEM (n=6). ***P<0.001 when compared to None. (B) IC₅₀ of the average inhibitory effect of Dibifree on MG induced glycation was determined.

FIG. 8 shows the effects of Dibifree on GLP-1 secretion. (A) GLP-1 secretion of intestinal stc-1 cells in the presence of Dibifree and (B) the viability of Dibifree treatment on stc-1 cells. Data represents as Mean±SEM (n=4). *P<0.05 and **P<0.01 when compared to None.

FIG. 9 shows the effect of Dibifree on DPP4 enzyme activity. (A) The effect of Dibifree on DPP4 enzyme activity and (B) IC₅₀ of the average inhibitory activity from Dibifree. Data represents as Mean±SEM (n=4). *P<0.05 and ***P<0.001 when compared to None.

FIG. 10 shows the results in terms of Glucose (AC) (Glucose Ante Cibum (before meals)) of the clinical trial (for 3 months) and the post-trial (for 3 months). There was a clearance period of 1 month between the clinical trial and the post-trial periods, wherein the control group were treated with Placebo in the trial period, and with Dibifree in the post-trial period; and the Dibifree group were treated with Dibifree in both of the trial and post trial periods. *P<0.05, **P<0.01 and ***P<0.001 when compared to Control.

FIG. 11 shows the results in terms of HbA1c (Glycated Hemoglobin) of the clinical trial (for 3 months) and the post-trial (for 3 months). There was a clearance period of 1 month between the clinical trial and the post-trial periods, wherein the control group were treated with Placebo in the trial period, and with Dibifree in the post-trial period; and the Dibifree group were treated with Dibifree in both of the trial and post trial periods. *P<0.05, **P<0.01 and ***P<0.001 when compared to Control.

FIG. 12 provides the potential mechanisms of Dibifree based on the results of the experiments, demonstrating that Dibifree exhibits the efficacy in activating GLP-1, inhibiting DPP-4 and the formation of AGEs.

### DETAILED DESCRIPTION OF THE INVENTION

Unless otherwise defined herein, scientific and technical terms used herein have the meanings that are commonly understood by those of ordinary skill in the art.

The present invention provides a new approach for treating or reversing diabetes, particularly in a subject having insulin resistance, e.g., type 2 diabetes.

According to the invention, a method and a supplement or composition for treating or reversing diabetes, particularly insulin-independent diabetes, e.g., type 2 diabetes are provided.

The term “subject” as used therein refers to a person, multiple persons, an animal, or multiple animals.

The term “therapeutically effective amount” as used herein refers to the amount of the supplement of the present invention administered is of sufficient quantity to achieve the intended purpose, such as, in this case, to treat or reverse diabetes in a subject.

Pharmaceutical compositions can be prepared by mixing with optional physiologically or pharmaceutically acceptable carriers, including solvents, dispersion media, isotonic agents and the like. The carrier can be liquid, semi-solid or solid carriers. In some embodiments, carriers may be water, saline solutions or other buffers (such as serum albumin and gelatin), carbohydrates (such as monosaccharides, disaccharides, and other carbohydrates including glucose, sucrose, trehalose, mannose, mannitol, sorbitol, or dextrins), gel, lipids, liposomes, stabilizers, preservatives, antioxidants (including ascorbic acid and methionine), chelating agents (such as EDTA), salt forming counter-ions (such as sodium) or combinations thereof.

In the invention, any of the anti-diabetes drugs, substances or a peptide or protein, or a mixture thereof may be used in combination with the supplement according to the invention. The anti-diabetes drug may be, including but not limited to metformin, thiazolidinediones and etc.

According to the invention, the extracts contained in the supplement may be prepared by any standard or commonly used methods. For example, the extracts may be extracted with water or ethanol.

The invention is further illustrated by the following example, which should not be construed as further limiting.

### EXAMPLES

### Example 1. Connecting Disease, Pathways and Dibifree Signatures Through Big Data Analysis

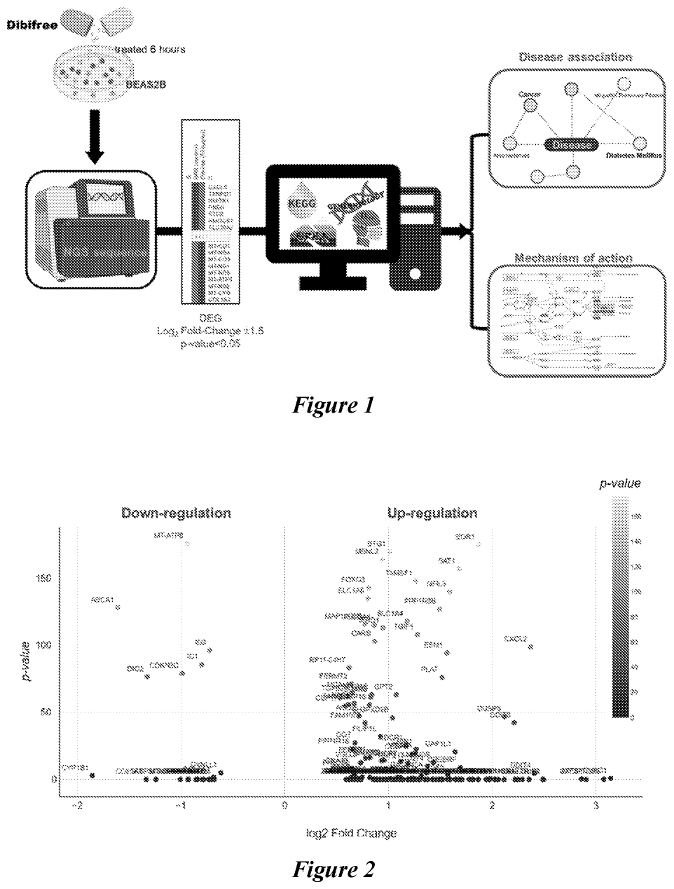

In order to elucidate the underlying mechanism of Dibifree for DM treatment, BEAS2B cells were treated with Dibifree and then total RNA samples were extracted and sequenced via NGS-RNAseq. The significant differential expression genes (DEGs) by Dibifree treatment were determined and enriched via KEGG, Gene Ontology (GO), Disease Ontology (DO) and GSEA for prediction of potential biological pathway moderated by the drug treatment. As demonstrated in FIG. 1, a bioinformatics pipeline has been applied to reveal underlying mechanism of Dibifree in treatment of DM.

In FIG. 1, the overview of the process connecting disease, pathways and Dibifree signatures through big data analysis is provided. NGS sequencing identified a list of DEGs with ±1.5 log 2 fold-change and p-value<0.05. Using enrichment analysis on DEGs and across four resources, we could predict the potential biological function of Dibifree. KEGG and GO provided the mechanism of action while GSEA and DO link a related-disease.

### Identify the Gene Expression Profile of Dibifree

Genetic expression profile played a key role to reveal the biological function and the connection to the disease of Dibifree. We treated Dibifree in BEAS2B, a normal lung bronchial epithelial cell, for 6 hours and extracted the total RNA through RNeasy® (QIAGEN). The significant differential expression genes (DEGs) between Dibifree treatment and control (PBS) were determined as log 2 fold change±1.5 and p-value less than 0.05 to select 373 up-regulated genes and 59 down-regulated genes. The volcano plot indicated the DEGs (p-value<0.001) by Dibifree treatment, such as CXCL8, EGR1, CXCL2, DIO2, and ABCA1, which were associated to diabetes or insulin response (see FIG. 2).

Dibifree was Connected to Current Diabetes Drug with Similar Functional Profile Via Connectivity Map/CLUE Platform.

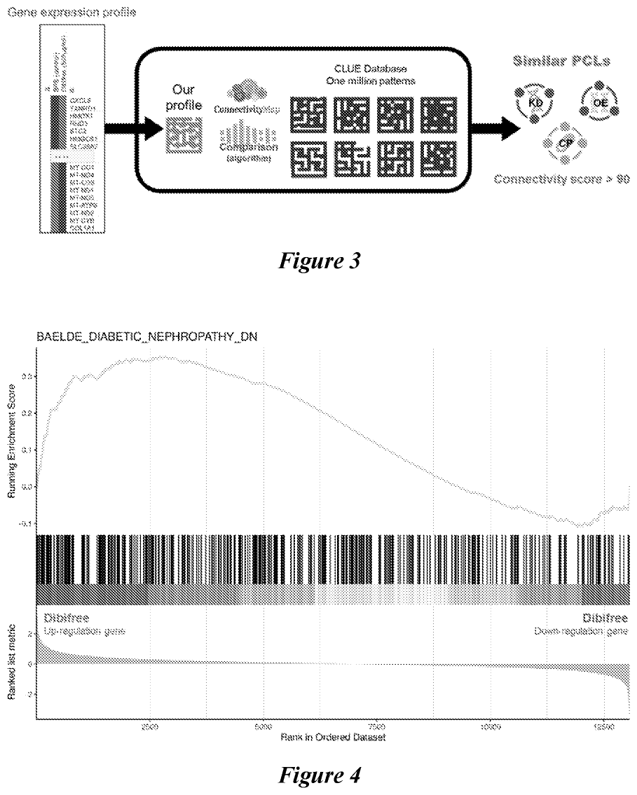

Based on the Connectivity Map/CLUE concept, we could connect compounds, diseases, and gene perturbagens using the gene expression list. The platform utilized a pattern comparison algorithm to connect 130 million profiles in the CLUE database. We input Dibifree DEG profile to match with CLUE reference profiles and to define the similarity. Connectivity scores above 90 has selected 17 PCLs (perturbagen classes) with high similarity with Dibifree biological function, which suggests potential mechanism of actions of the drug (see FIG. 3, Table 1), such as IKK inhibitor, BCL inhibitor, EGFR inhibitor, RAF inhibitor. In addition, the analysis reported that Dibifree might have weak similarity function with rosiglitazone indicated by a connectivity score of 61.72. On the other hand, Dibifree was not related to other diabetes drugs, including metformin, glibenclamide, or glimepiride (Table 2). Taken together, this analysis suggests that Dibifree has different mechanism of action compared to conventional diabetes drugs.

Using the pattern comparison algorithm of the Connectivity Map/CLUE platform, a list of similar compounds, and genetic perturbagens including over expression and knockdown genes could be obtained (see FIG. 3). The PCL was a cluster to define the similar mechanism of action among these compounds or genes; therefore, PCLs implied the possible function of Dibifree (see Table 1). Using the compound comparison, we could imply that Dibifree might have distinct gene expression signatures (or different mechanism of actions) to other diabetes drugs (see Table 2).

TABLE 1
17 PCLs with connectivity scores higher than 90
suggest potential Dibifree mechanism of actions.

| PCL name | score |
|---|---|
| Vesicular Transport LOF | 99.54 |
| PKC activator | 99.18 |
| PKC inhibitor | 99.11 |
| ATP synthase inhibitor | 99.03 |
| T-type calcium channel blocker | 98.98 |
| IKK inhibitor | 98.61 |
| ATPase inhibitor | 98.48 |
| BCL inhibitor | 98.35 |
| Heat shock 70 kDa proteins LOF | 96.94 |
| Estrogen receptor antagonist | 96.13 |
| Tubulin inhibitor | 94.79 |
| EGFR inhibitor | 94.33 |
| HIF activator | 94.13 |
| V type ATPases LOF | 94 |
| Proteasome inhibitor | 93.73 |
| RAF inhibitor | 90.53 |
| FLT3 inhibitor | 90.34 |

TABLE 2
The connectivity score of between Dibifree gene
expression profile and that of insulin sensitizers
and gluconeogenesis inhibitors via CLUE.

| Name | Type | Score |
|---|---|---|
| rosiglitazone | CP | 61.72 |
| GAA (α-Glucosidase ) | KD | 11.62 |
| pioglitazone | CP | 6.59 |
| troglitazone | CP | −0.42 |
| DPP4 | KD | −2.12 |
| ciglitazone | CP | −5.91 |
| glibenclamide | CP | −6.5 |
| GW-0742 | CP | −6.95 |
| metformin | CP | −7.24 |
| CAY-10415 | CP | −16.31 |
| GW-501516 | CP | −17.38 |
| glimepiride | CP | −24.61 |
| ZD-2079 | CP | −29.25 |
| glimepiride | CP | −34.4 |
| GW-1929 | CP | −52.56 |
| methazolamide | CP | −58.45 |

Uncover the biological connection of diseases and Dibifree through a series of bioinformatics pipeline.

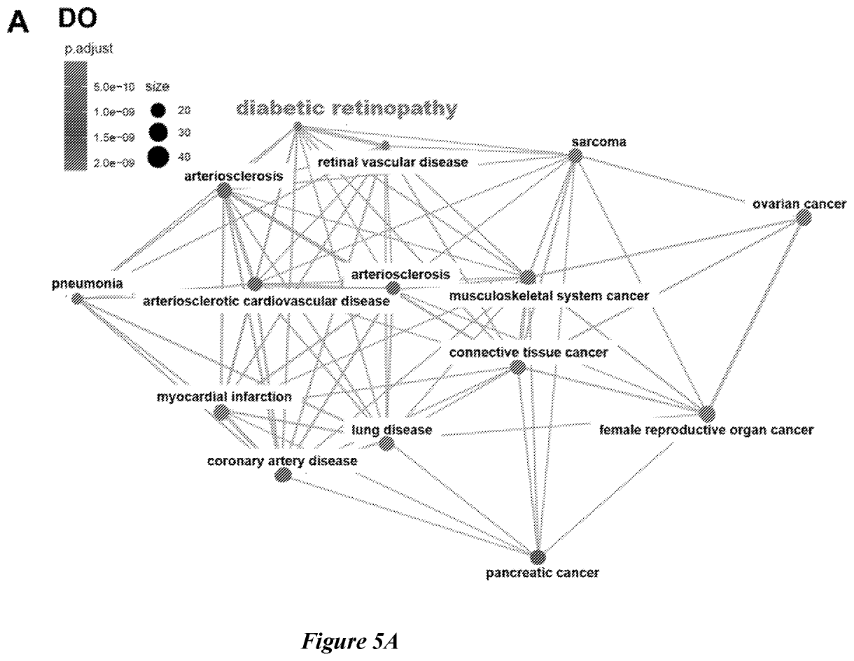

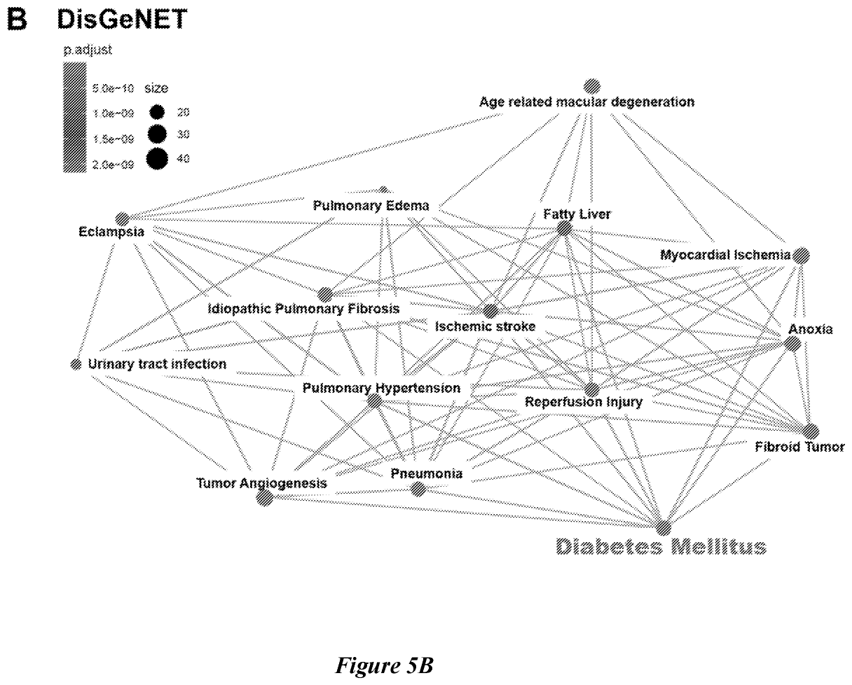

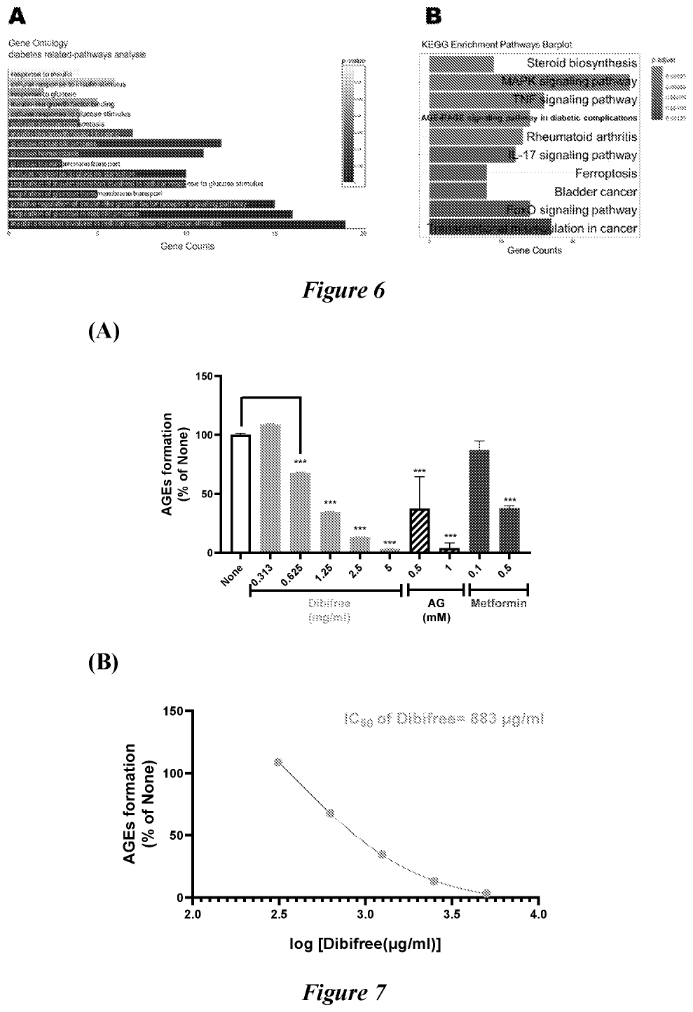

Since we have acquired the Dibifree treatment response profile, we could uncover a possible mechanism of action to connect to the disease. GSEA analysis showed that Dibifree could up-regulate genes, which were down-regulated in kidneys of patients with T2DM, suggesting the drug might reverse the diabetic signature (see FIG. 4). In addition, Disease Ontology analysis strengthened the evidence of the Dibifree treatment on DM. Both DO (see FIG. 5(A)) and DisGeNET (see FIG. 5(B)) two databases predicted the connection among Dibifree, DM, comorbidities, and complications. The network showed the top 15 associated diseases, including diabetic retinopathy, fatty liver, arteriosclerotic cardiovascular disease, coronary artery disease, pancreatic cancer, pneumonia, and so on comorbidities. GO enrichment analysis for the Dibifree profile also showed diabetes-related pathways with significant enrichment, for example, insulin-like growth factor binding, glucose homeostasis, and regulation of insulin secretion involved in cellular response to glucose stimulus (FIG. 6(A)). On the other hand, enrichment using KEGG also revealed that Dibifree might regulate AGE-RAGE signaling pathway in diabetic complications, insulin resistance, and microRNAs in cancer (FIG. 6(B)). Taken together, Dibifree might exhibit a novel mechanism of action for diabetes treatment.

### Example 3 Study for Mechanism of Action

### Methods

### Methylglyoxal (MG) Induced Advanced Glycation Endproducts (AGEs) Formation

Methylglyoxal (MG) induced glycation of bovine serum albumin (BSA) was employed to evaluate anti-glycation effect of Dibifree. Fluorescence levels of glycated BSA were measured. Briefly, BSA (10 mg/ml) was non-enzymatically glycated via incubation in 1 M PBS, pH 7.4, at 37° C. for 7 days in the presence of 1 mM MG and 3 mM sodium azide. The test reagent is tested at various concentrations. Fluorescence of the samples was measured at the excitation and emission wavelengths of 335 and 385 nm, respectively, versus a blank containing the protein and MG. The % inhibition by different concentrations of samples was calculated according to the following equation: [1−(Fsample+BSA+glucose−Fsample+BSA/FBSA+glucose−FBSA)]×100. Aminoguanidine (AG) was used as a positive control. Aminoguanidine (AG) and metformin were used as anti-glycation reference drugs.

### Acute GLP-1 Secretion Assay

The murine intestinal secretin tumor cell line (STC-1) cells were routinely grown as a monolayer with DMEM containing 10% fetal bovine serum and 5% penicillin and streptomycin mixture in culture dishes at 37° C. under 5% CO2/air with 90% humidity. STC-1 cells were used for GLP-1 secretion test. Briefly, cultured cells were seeded into 24-well plate (0.5×10⁵ cells/well) and allowed for overnight attachment. When cells reach to the confluence 48 h after, they were washed with KRBB (1.1 mM glucose and 0.1% BSA) for three times and incubated for 30 min at 37° C. Wash medium was then exchanged with Dibifree containing medium made with KRBB (16.7 mM glucose) for 60 min incubation at 37° C., which was then collected and stored at −20° C. GLP-1 Active level in each sample was measured by an HTRF assay (Cisbio).

### DPP4 Enzyme Activity Assay

The inhibitory effect of Dibifree on DPP-4 was analyzed using a fluorometric assay kit (BioVision, Milpitas, CA, USA) according to manufacturer's instructions. Mouse serum was prepared as DPP-4 enzyme source. Serum DPP-4 activity in the presence of vehicle or rutin was determined after 30 min incubation at 37° C.

### Statistics

Data were presented as means±standard error of the mean. Statistical analyses were performed using GraphPad Prism (GraphPad, San Diego, CA, USA). Single parameter-based comparisons were made using one way ANOVA with Dunnett's multiple comparisons test. P values less than 0.05, 0.01, and 0.001 were considered to be significant.

### Results

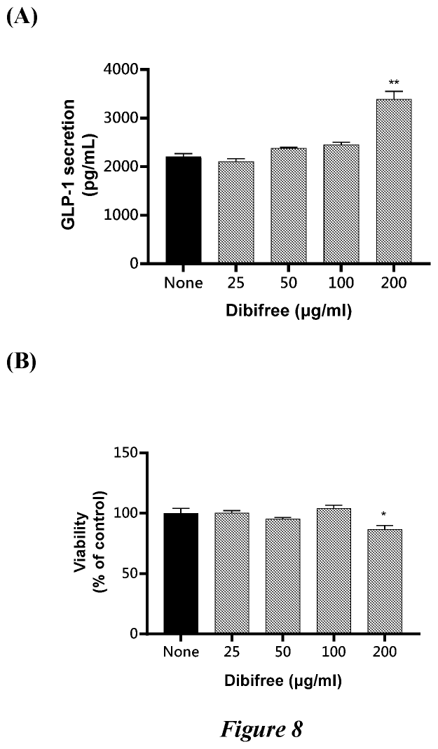

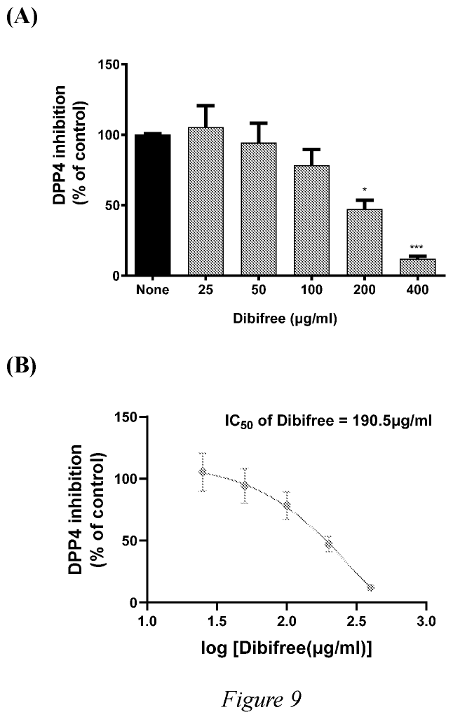

The results are shown in FIGS. 7-9. As shown in FIG. 7, the inhibitory effect of Dibifree on MG induced glycation was significant as compared to the None group (control). The effects of Dibifree on GLP-1 secretion and the viability of Dibifree treatment on stc-1 cells were significant as compared to the None group (control), see FIG. 8. Further, The effect of Dibifree on DPP4 enzyme activity was significant as compared to the None group (control), see FIG. 9.

### Example 3. Clinical Trial

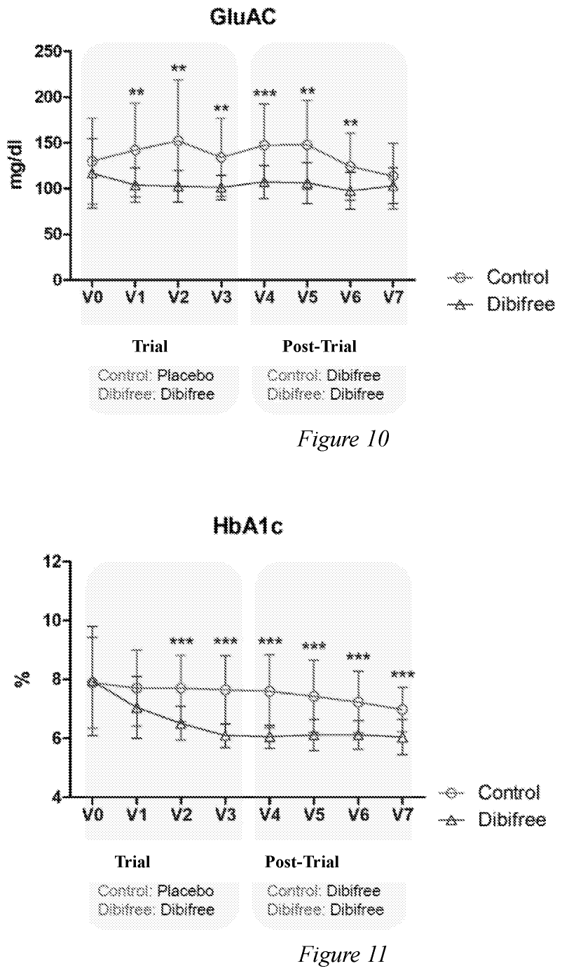

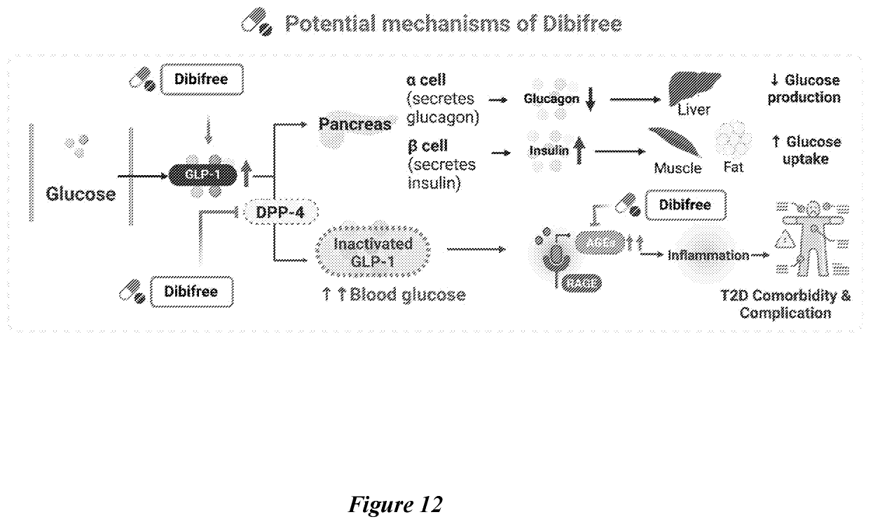

A clinical trial with 40 patients suffering from type 2 diabetes was conducted during August 2020 to July 2021. There was a clearance period of 1 month between the clinical trial and the post-trial periods, wherein the control group were treated with Placebo in the trial period, and with Dibifree in the post-trial period; and the Dibifree group were treated with Dibifree in both of the trial and post-trial periods. The results were given in FIGS. 10 and 11.

As shown in FIG. 10, in the trial and post-trial periods, the average levels of Glucose (AC) (Glucose Ante Cibum (before meals)) of the Dibifree group were significantly lower than those of the control group. However, the levels of Glucose (AC) of the control group were decreased at the end of the post-trial period after the three-month treatment with Dibifree.

As shown in FIG. 11, in the trial and post-trial periods, the average levels of HbA1c (Glycated Hemoglobin) of the Dibifree group were significantly lower than those of the control. However, the levels of HbA1c of the control group were decreased at the end of the post-trial period after the three-month treat with Dibifree.

In view of the above, Dibifree is effective in treating and even reversing diabetes, particularly type 2 diabetes, through activating GLP-1, inhibiting DPP4 and inhibiting the formation of AGEs. Dibifree can regulate AGE-RAGE signaling pathway in diabetic complications, insulin resistance, and microRNAs in cancer, and exhibits a novel mechanism of action for diabetes treatment.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

1. A use of a supplement for manufacturing a medicament for treating diabetes, wherein the supplement comprises of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

2. The use of claim 1, wherein the supplement consists essentially of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

3. The use of claim 1, wherein the supplement consists of 5 wt %-30 wt % Green coffee (Coffea arabica) bean extract, 5 wt %-30 wt % Bitter melon (Momordica charantia) fruit extract, 5 wt %-30 wt % Celery (Apium graveolens) seed extract, 5 wt %-30 wt % Bakers yeast (Saccharomyces cerevisiae) cell wall extract, 5 wt %-30 wt % Acerola (Malpighia emarginata) fruit extract, 5 wt %-30 wt % Grape (Vitis vinifera) seed extract, 5 wt %-30 wt % Green tea leaf extract, and 5 wt %-30 wt % Hydrolyzed soy protein powder, based on the total weight of the supplement, respectively.

4. The use of claim 1, wherein the Bitter melon (Momordica charantia) fruit extract is at 12.5 wt %, Celery (Apium graveolens) seed extract is at 12.5 wt %, Bakers yeast (Saccharomyces cerevisiae) cell wall extract is at 12.5 wt %, Acerola (Malpighia emarginata) fruit extract is at 12.5 wt %, Grape (Vitis vinifera) seed extract is at 12.5 wt %, Green tea leaf extract is at 12.5 wt %, Hydrolyzed soy protein powder is at 12.5 wt %, based on the total weight of the supplement, respectively.

5. The use of claim 1, wherein the supplement exhibits the efficacy in activating Glucagon-like peptide-1 (GLP-1), inhibiting dipeptidyl peptidase-4 (DPP4), and inhibiting formation of Advanced glycation end products (AGEs).

6. A use of the supplement set forth in claim 1 in manufacturing a medicament for reversing diabetes in a subject, wherein the supplement at a therapeutically effective amount to activate Glucagon-like peptide-1 (GLP-1), inhibit Dipeptidyl peptidase-4 (DPP4) and/or inhibit the formation of Advanced glycation end products (AGEs) in the subject.

7. A use of the supplement set forth in claim 1 for manufacturing a medicament for treating or reversing diabetes.

8. The use of claim 1, wherein the diabetes is type 2 diabetes.

9. The use of claim 1, wherein the diabetes is insulin-resistance diabetes.

10. A pharmaceutical composition for treating or reversing diabetes in a subject comprises of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

11. The pharmaceutical composition of claim 10, wherein the pharmaceutical composition consists essentially of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

12. The pharmaceutical composition of claim 10, wherein the pharmaceutical composition consists of 5%-30% Bitter melon (Momordica charantia) fruit extract, 5%-30% Celery (Apium graveolens) seed extract, 5%-30% Bakers yeast (Saccharomyces cerevisiae) cell wall extract, 5%-30% Acerola (Malpighia emarginata) fruit extract, 5%-30% Grape (Vitis vinifera) seed extract, 5%-30% Green tea leaf extract, and 5%-30% Hydrolyzed soy protein powder, based on the total weight of the supplement, respectively.

13. The pharmaceutical composition of claim 10, wherein the Bitter melon (Momordica charantia) fruit extract is at 12.5 wt %, Celery (Apium graveolens) seed extract is at 12.5 wt %, Bakers yeast (Saccharomyces cerevisiae) cell wall extract is at 12.5 wt %, Acerola (Malpighia emarginata) fruit extract is at 12.5 wt %, Grape (Vitis vinifera) seed extract is at 12.5 wt %, Green tea leaf extract is at 12.5 wt %, Hydrolyzed soy protein powder is at 12.5 wt %, based on the total weight of the supplement, respectively.

14. The pharmaceutical composition of claim 10, in which the diabetes is type 2 diabetes.

15. The pharmaceutical composition of claim 10, in which the diabetes is insulin-resistance diabetes.

16. A combination or composition for treating or reversing diabetes which comprises a therapeutically effective amount of the supplement set forth in claim 1, and an anti-diabetes drug, a substance, a peptide, a protein, or a mixture thereof.

17. The combination or composition of claim 16, wherein the diabetes is type 2 diabetes.

18. The combination or composition of claim 16, wherein the diabetes is insulin-resistance diabetes.
